(19) United States
(12) Patent Application Publication
(10) Pub. No.: US 2026/0078167 A1
(43) Pub. Date: Mar. 19, 2026

(54) FUSION PROTEIN

(71) Applicant: LEVICEPT LIMITED, Sandwich, Kent (GB)

(72) Inventors: Jason RUTT, Sandwich (GB); Simon WESTBROOK, Sandwich, Kent (GB)

(21) Appl. No.: 19/397,512

(22) Filed: Nov. 21, 2025

Related U.S. Application Data
(63) Continuation of application No. PCT/GB2024/051315, filed on May 21, 2024.

(30) Foreign Application Priority Data
May 26, 2023 (GB) 2307932.0

(51) Int. Cl. C07K 14/71; A61K 38/00; A61P 19/02; A61P 29/02

(52) U.S. Cl. CPC C07K 14/71; A61P 19/02; A61P 29/02; A61K 38/00; C07K 2319/30

(57) ABSTRACT

The present invention relates to a p75NTR neurotrophin binding protein (NBP)-Fc fusion protein comprising a p75NTR(NBP) portion and an immunoglobulin portion. In certain embodiments, the p75NTR(NBP)-Fc fusion protein is for use in the treatment of pain and/or a symptom of pain.

This application is a bypass continuation of International Application No. PCT/GB2024/051315, filed May 21, 2024, which claims the benefit of priority of United Kingdom Application No. 2307932.0, filed May 26, 2023, the disclosures of each are hereby incorporated by reference as if written herein in their entireties.

### INCORPORATION OF SEQUENCE LISTING

The sequence listing that is contained in the file named “P234.011WO,” which was created on May 17, 2024, and which has a file size of 27,197 bytes as measured in Microsoft Windows operating system, is filed electronically herewith and incorporated herein by reference.

### BACKGROUND TO THE INVENTION

The neurotrophins, neurotrophic growth factor (NGF), brain-derived neurotrophic factor (BDNF), neurotrophin 3 (NT-3), and neurotrophin 4/5 (NT-4/5) act via four receptors: the low affinity p75 neutrophic receptor (p75NTR), and the high affinity tyrosine kinase receptors; TrkA, TrkB, and TrkC. The low affinity receptor p75NTR binds and is activated by all four neurotrophins and has been reported to function independently from the other receptors. However, the Trk receptors are more selectively activated i.e. NGF is the selective ligand for TrkA, BDNF the ligand for TrkB and NT-3, 4/5 the ligands for TrkC. In addition it has been reported, when p75NTR and Trk proteins are co-expressed, they form complexes, which alter the signaling of both receptors (Huang and Reichardt, 2003, Annu Rev Biochem. 72:609-42). Indeed, it has been suggested that p75NTR facilitates the selectivity of each of the neutrophins for their respective Trk receptor.

The p75NTR is a member of the tumor necrosis factor receptor superfamily (TNFR-SF) and was the first member of this superfamily to be characterized fully. The superfamily (encoded by some 30 genes in humans) is defined by ligand-binding domains consisting of one or more (typically four) repeats of a 40 amino acid cysteine-rich domain (CRD) that was first identified in p75NTR (Johnson et al., 1986 Cell 47:545-554; Radeke et al., 1987 Nature 325:593-597). In contrast, no sequence motif is shared by the intracellular domains of all TNFR-SF family members. Consequently, signaling mechanisms of TNFR-SF proteins vary significantly.

An unusual feature of p75NTR structure is the existence of a disulfide-linked p75NTR dimer, formed via cysteinyl residues within the transmembrane domains. This disulfide linkage is required for effective neurotrophin-dependent signaling by p75NTR and plays an important role in the formation of an intracellular and extracellular domain (Vilar et al., 2009 Neuron 62:72-83). Neurotrophins exist physiologically as noncovalently associated dimers (Bothwell and Shooter, 1977 J Biol Chem. 252(23):8532-6) with a distribution half-life of approximately 5 min (Tria et al., 1994 Exp Neurol. 127(2):178-83). Neurotrophin-dependent p75NTR activation involves association of a neurotrophin dimer with CRDs 2-4 of the two extracellular domains of a p75NTR dimer (He and Garcia, 2004 Science 304:870-875). Recent studies support a model in which neurotrophin binding causes the two extracellular domains of p75NTR dimers to move closer together, forcing the intracellular domains to splay apart in a snail-tong-like motion centered on the disulfide bond and permitting association of the intracellular domains with the signaling adapter proteins, NRIF and TRAF6 (Vilar et al., 2009 J Cell Sci 122:3351-3357, Vilar et al., 2009 Neuron 62:72-83). Intra-transmembrane domain disulfide bonds, such as are present in p75NTR, have not been described previously in other TNFR-SF family members, or in any other membrane protein.

p75NTR undergoes sequential proteolytic cleavage by □-secretase and □-secretase activities and matrix metalloproteinases (MMPs), releasing its intracellular domain (ICD) into the cytoplasm, in a manner analogous to the cleavage-dependent signaling pathway of Notch and □-amyloid precursor protein (Jung et al., 2003 J Biol Chem 278:42161-42169; Kanning et al., 2003 J Neuro-sci 23:5425-5436). Cytoplasmic release of the p75NTR ICD by this pathway promotes signaling by associated NRIF (Kenchappa et al., 2006 Neuron 50:219-232). The role of the extracellular domain of p75NTR, following the proteolytic cleavage by □-secretase and □-secretase activities and MMPs isn't fully understood.

It has been documented that NGF and other neurotrophins (BDNF, NT-3 and NT-4/5) play a significant role in pathology for example pain due to osteoarthritis, pancreatitis, rheumatoid arthritis, psoriasis, pruritis and multiple sclerosis (Watanabe et al., 2008 J Neurosci Res. 86(16):3566-74; Raychaudhuri et al., 2011 Arthritis Rheum. 63(11):3243-52; Barthel et al., 2009 Arthritis Res Ther. 11(3):R82; Truzzi et al., 2011 Cell Death Differ. 18:948-58; McDonald et al., 2011 Curr Med Chem. 18:234-44; Yamaoka et al., 2007 J Dermatol Sci. 46(1):41-51). It was been demonstrated that selective antibodies to any of the neutrophins; either NGF or BDNF, NT-3 and NT-4/5 significantly reduce pain. Furthermore, antibodies directed to the neurotrophin receptors p75NTR Trk A, Trk B or Trk C have also been demonstrated to be efficacious in models of pain (Orita S et al., 2010 J Orthop Res. 28:1614-20; Svensson P et al., 2010 Pain. 148:473-80; Iwakura et al., 2010 J Hand Surg Am. 35:267-73; Cirilio et al., 2010 Cell Mol Neurobiol. 30:51-62; Pezet et al., 2010 Pain. 90:113-25; Hayashi et al., 2011 J Pain. 12:1059-68; Chu et al., 2011 Pain. 152:1832-7; Ueda et al., 2010 J Pharmacol Sci.; 112:438-43; Ghilardi et al., 2010 Bone. 48:389-98; Fukui et al., 2010 J Orthop Res. 2010; 28:279-83). Fukui et al., (2010) in a model of pain (mechanical allodynia following sciatic nerve crush) demonstrated significant efficacy on pain related endpoints following treatment with an anti-p75NTR antibody. It was concluded from this study that the treatment with a p75NTR inhibitory antibody reduced CGRP and p75NTR expression resulting in a significant reduction in pain.

The current invention relates to a p75NTR neurotrophin binding protein (NBP)-Fc fusion protein. We describe the affinity and in vivo kinetics of such a molecule, as well as efficacy in the treatment of pain in an animal model. The p75NTR(NBP)-Fc fusion protein finds use in the treatment of pain and other neurotrophic factor related pathologies such as psoriasis, eczema, rheumatoid arthritis, cystitis, endometriosis and osteoarthritis.

### BRIEF DESCRIPTION OF THE INVENTION

According to a first aspect of the present invention there is provided a p75NTR neurotrophin binding protein (NBP)-Fc fusion protein, comprising:
(a) a p75NTR(NBP) portion selected from one of Seq ID no 1-19; and
(b) an immunoglobulin Fc portion selected from one of Seq ID no 20-24

Wherein, the p75NTR(NBP) and Fc portions are connected via a linker, the linker comprises a peptide of formula Gₓ, where x is 1, 2, 3, 4, 5 or 6

In a preferred embodiment wherein the linker does not comprise or consist of the sequence GGGGS.

In a further embodiment the linker is GGG

In a particularly preferred embodiment of the p75NTR(NBP)-Fc fusion protein according to the invention, the Fc region is an Fc suitable for human use.

In yet another preferred embodiment, the p75NTR(NBP) portion of the fusion protein of the invention comprises or consists of the amino acid sequence set forth in SEQ ID NO. 1. In another preferred embodiment, the p75NTR(NBP) portion of the fusion protein of the invention comprises or consists of the amino acid sequence set forth in SEQ ID No. 2. In another preferred embodiment, the p75NTR(NBP) portion of the fusion protein of the invention comprises or consists of the amino acid sequence set forth in SEQ ID No. 3. In another preferred embodiment, the p75NTR(NBP) portion of the fusion protein of the invention comprises or consists of the amino acid sequence set forth in SEQ ID No. 4. In another preferred embodiment, the p75NTR(NBP) portion of the fusion protein of the invention comprises or consists of the amino acid sequence set forth in SEQ ID No. 5. In another preferred embodiment, the p75NTR(NBP) portion of the fusion protein of the invention comprises or consists of the amino acid sequence set forth in SEQ ID No. 6. In another preferred embodiment, the p75NTR(NBP) portion of the fusion protein of the invention comprises or consists of the amino acid sequence set forth in SEQ ID No. 7. In another preferred embodiment, the p75NTR(NBP) portion of the fusion protein of the invention comprises or consists of the amino acid sequence set forth in SEQ ID No. 8. In another preferred embodiment, the p75NTR(NBP) portion of the fusion protein of the invention comprises or consists of the amino acid sequence set forth in SEQ ID No. 9. In another preferred embodiment, the p75NTR(NBP) portion of the fusion protein of the invention comprises or consists of the amino acid sequence set forth in SEQ ID No. 10. In another preferred embodiment, the p75NTR(NBP) portion of the fusion protein of the invention comprises or consists of the amino acid sequence set forth in SEQ ID No. 11. In another preferred embodiment, the p75NTR(NBP) portion of the fusion protein of the invention comprises or consists of the amino acid sequence set forth in SEQ ID No. 12. In another preferred embodiment, the p75NTR(NBP) portion of the fusion protein of the invention comprises or consists of the amino acid sequence set forth in SEQ ID No. 13. In another preferred embodiment, the p75NTR(NBP) portion of the fusion protein of the invention comprises or consists of the amino acid sequence set forth in SEQ ID No. 14. In another preferred embodiment, the p75NTR(NBP) portion of the fusion protein of the invention comprises or consists of the amino acid sequence set forth in SEQ ID No. 15. In another preferred embodiment, the p75NTR(NBP) portion of the fusion protein of the invention comprises or consists of the amino acid sequence set forth in SEQ ID No. 16. In another preferred embodiment, the p75NTR(NBP) portion of the fusion protein of the invention comprises or consists of the amino acid sequence set forth in SEQ ID No. 17. In another preferred embodiment, the p75NTR(NBP) portion of the fusion protein of the invention comprises or consists of the amino acid sequence set forth in SEQ ID No. 18. In another preferred embodiment, the p75NTR(NBP) portion of the fusion protein of the invention comprises or consists of the amino acid sequence set forth in SEQ ID No. 19.

In another preferred embodiment, the Fc portion of the fusion protein of the invention comprises or consists of the amino acid sequence set forth in SEQ ID No. 20. In another preferred embodiment, Fc portion of the fusion protein of the invention comprises or consists of the amino acid sequence set forth in SEQ ID No. 21. In another preferred embodiment, the Fc portion of the fusion protein of the invention comprises or consists of the amino acid sequence set forth in SEQ ID No. 22. In another preferred embodiment, the Fc portion of the fusion protein of the invention comprises or consists of the amino acid sequence set forth in SEQ ID No. 23. In another preferred embodiment, the Fc portion of the fusion protein of the invention comprises or consists of the amino acid sequence set forth in SEQ ID No. 24.

In a preferred embodiment, the p75NTR(NBP)-Fc fusion protein according to the invention binds to any of NGF, BDNF, NT3 or NT4/5 with a binding affinity (K_{d}) of between about 0.01 nM to about 50 nM as measured by surface plasmon resonance at 20° C.

In second aspect of the present invention, the p75NTR(NBP)-Fc fusion protein as described according to any other aspect of the invention is provided for use in the treatment of pain.

In a third aspect of the present invention, there is provided a nucleic acid molecule encoding the p75NTR(NBP)-Fc fusion protein according to the first or second aspects of the invention, optionally further comprising encoding a signal sequence.

In a fourth aspect of the present invention, there is provided a replicable expression vector for transfecting a cell, optionally a mammalian cell, the vector comprising the nucleic acid molecule according to the third aspect of the present invention.

Preferably, the replicable expression vector is a viral vector.

In a fifth aspect of the present invention, there is provided a host cell harbouring the nucleic acid molecule of the third aspect of the invention.

In a sixth aspect of the present invention, the nucleic acid molecule according to the third aspect of the invention or the vector according to the fourth aspect of the present invention is for use in the treatment of pain.

Pain includes but is not limited to: acute pain; chronic pain; inflammatory pain; nociceptive pain; neuropathic pain; hyperalgesia; allodynia; central pain; cancer pain; post-operative pain; visceral pain; musculo-skeletal pain; heart or vascular pain; head pain including migraine; orofacial pain, including dental pain; and back pain. Treatment of pain includes, but is not limited to, preventing, ameliorating, controlling, reducing incidence of, or delaying the development or progression of pain.

In a seventh aspect of the present invention, the nucleic acid molecule according to the third aspect of the invention or the vector according to the fourth aspect of the present invention is for use in the treatment of osteoarthritis.

In an eighth aspect, there is provided the p75NTR(NBP)-Fc fusion protein according to the first or second aspects, or the nucleic acid or vector according to the third or fourth aspect, wherein the p75NTR(NBP)-Fc fusion protein or nucleic acid molecule or vector is for separate, sequential or simultaneous use in a combination combined with a second pharmacologically active compound.

In a ninth aspect, the present invention provides a pharmaceutical composition, comprising the p75NTR(NBP)-Fc fusion protein according to any aspect of the invention or the nucleic acid molecule or vector according to any aspect of the invention, and a pharmaceutically acceptable carrier and/or an excipient.

Preferably, the pharmaceutical composition is for use in any one or more of preventing, ameliorating, controlling, reducing incidence of, or delaying the development or progression of osteoarthritis in mammals,

In one embodiment the mammal is a human

In another embodiment the mammal is an animal, preferably a dog, cat, elephant or horse. In one particularly preferred embodiment the animal is dog.

In a further aspect of the present invention, there is provided a kit comprising:
(a) the p75NTR(NBP)-Fc fusion protein according to any aspect of the present invention, or the nucleic acid molecule or vector according any aspect of the present invention, or the pharmaceutical composition according to the eighth aspect; and
(b) instructions for the administration of an effective amount of said the p75NTR(NBP)-Fc fusion protein, nucleic acid molecule, vector or pharmaceutical composition to an animal for any one or more of the prevention or treatment of pain or for ameliorating, controlling, reducing incidence of, or delaying the development or progression of pain.

In yet another aspect of the present invention there is provided a method of treating and or preventing pain in an animal comprising administering to said individual a therapeutically effective amount of the p75NTR(NBP)-Fc fusion protein according to any aspect of the invention, or the nucleic acid molecule or vector according to any aspect of the invention, optionally further comprising a pharmaceutically acceptable carrier, or the pharmaceutical composition according to the eighth aspect of the invention.

### DESCRIPTION OF THE FIGURES

FIG. 1. Amino acid sequence of a p75NTR(NBP) portion of the fusion protein according to the present invention (SEQ ID No. 1).

FIG. 2. Amino acid sequence of a p75NTR(NBP) portion of the fusion protein according to the present invention (SEQ ID No. 2).

FIG. 3. Amino acid sequence of a p75NTR(NBP) portion of the fusion protein according to the present invention (SEQ ID No. 3).

FIG. 4. Amino acid sequence of a p75NTR(NBP) portion of the fusion protein according to the present invention (SEQ ID No. 4).

FIG. 5. Amino acid sequence of a p75NTR(NBP) portion of the fusion protein according to the present invention (SEQ ID No. 5).

FIG. 6. Amino acid sequence of a p75NTR(NBP) portion of the fusion protein according to the present invention (SEQ ID No. 6).

FIG. 7. Amino acid sequence of a p75NTR(NBP) portion of the fusion protein according to the present invention (SEQ ID No. 7).

FIG. 8. Amino acid sequence of a p75NTR(NBP) portion of the fusion protein according to the present invention (SEQ ID No. 8).

FIG. 9. Amino acid sequence of a p75NTR(NBP) portion of the fusion protein according to the present invention (SEQ ID No. 9).

FIG. 10. Amino acid sequence of a p75NTR(NBP) portion of the fusion protein according to the present invention (SEQ ID No. 10).

FIG. 11. Amino acid sequence of a p75NTR(NBP) portion of the fusion protein according to the present invention (SEQ ID No. 11).

FIG. 12. Amino acid sequence of a p75NTR(NBP) portion of the fusion protein according to the present invention (SEQ ID No. 12).

FIG. 13. Amino acid sequence of a p75NTR(NBP) portion of the fusion protein according to the present invention (SEQ ID No. 13).

FIG. 14. Amino acid sequence of a p75NTR(NBP) portion of the fusion protein according to the present invention (SEQ ID No. 14).

FIG. 15. Amino acid sequence of a p75NTR(NBP) portion of the fusion protein according to the present invention (SEQ ID No. 15).

FIG. 16. Amino acid sequence of a p75NTR(NBP) portion of the fusion protein according to the present invention (SEQ ID No. 16).

FIG. 17. Amino acid sequence of a p75NTR(NBP) portion of the fusion protein according to the present invention (SEQ ID No. 17).

FIG. 18. Amino acid sequence of a p75NTR(NBP) portion of the fusion protein according to the present invention (SEQ ID No. 18).

FIG. 19. Amino acid sequence of a p75NTR(NBP) portion of the fusion protein according to the present invention (SEQ ID No. 19).

FIG. 20. Amino acid sequence of a suitable human Fc portion of the fusion protein according to the present invention (SEQ ID No. 20).

FIG. 22. Amino acid sequence of Cat IgG-Fc portion of the fusion protein according to the present invention (SEQ ID No. 21).

FIG. 22. Amino acid sequence of horse IgG-Fc portion of the fusion protein according to the present invention (SEQ ID No. 22).

FIG. 23. Amino acid sequence of Cat IgG-Fc portion of the fusion protein according to the present invention (SEQ ID No. 23).

FIG. 24: Amino acid sequence of Cat IgG-Fc portion of the fusion protein according to the present invention (SEQ ID No. 24).

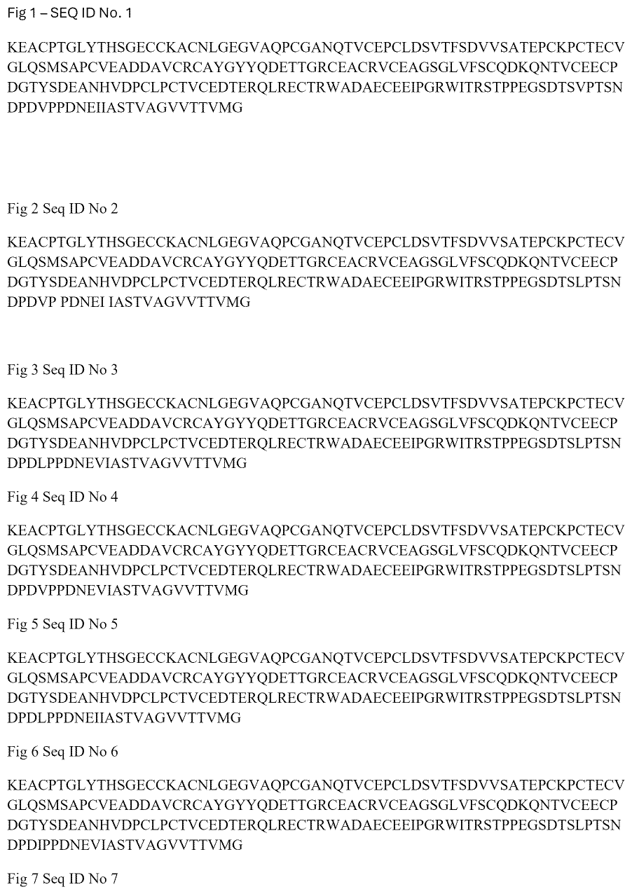

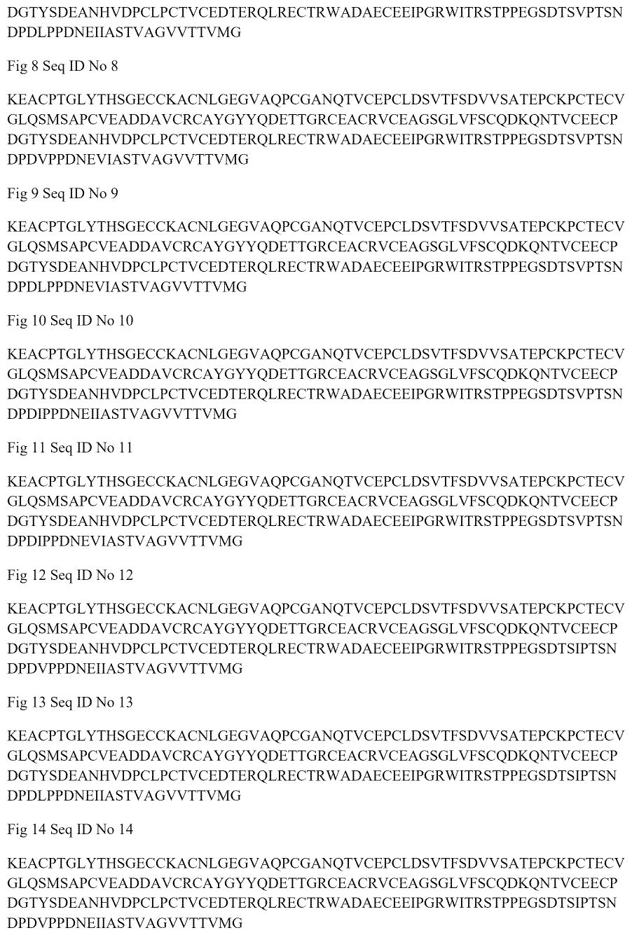

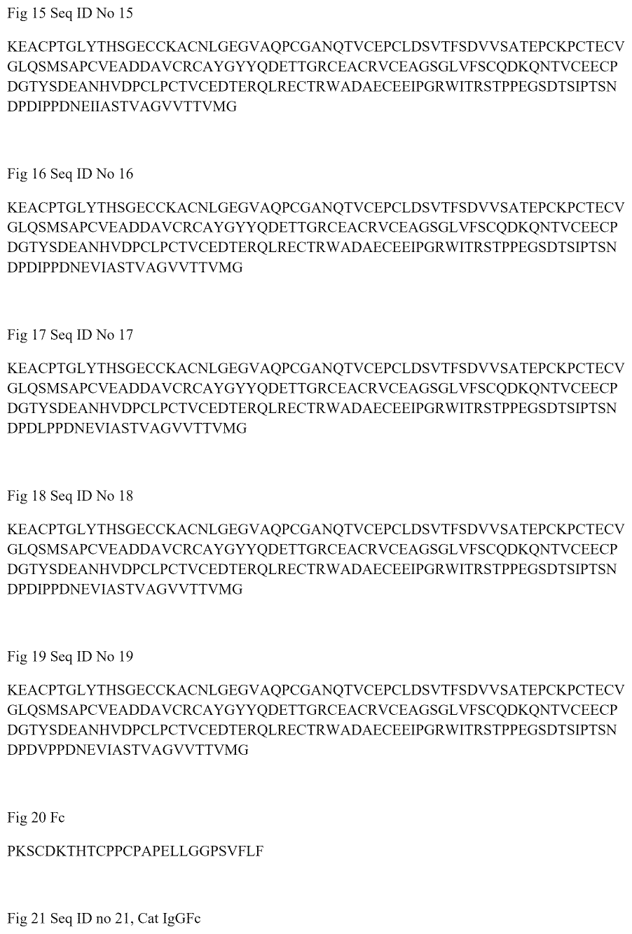

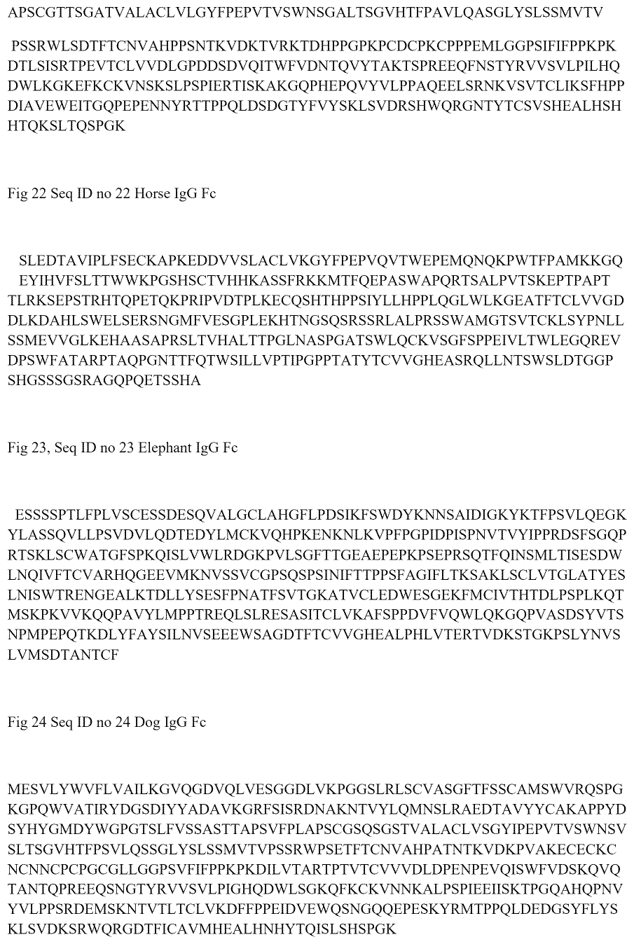

### DETAILED DESCRIPTION OF THE INVENTION

According to a first aspect of the present invention there is provided a p75NTR neurotrophin binding protein (NBP)-Fc fusion protein, comprising:
(a) a p75NTR(NBP) portion selected from one of Seq ID no 1-19; and
(b) an immunoglobulin Fc portion selected from one of Seq ID no 20-24.

Wherein, the p75NTR(NBP) and Fc portions are connected via a linker, the linker comprises a peptide of formula Gₓ, where x is 1, 2, 3, 4, 5 or 6

G is Glycine.

In a preferred embodiment wherein the linker does not comprise or consist of the sequence GGGGS.

In a further embodiment the linker is GGG

In a particularly preferred embodiment of the p75NTR(NBP)-Fc fusion protein according to the invention, the Fc region is an Fc suitable for human use.

In yet another preferred embodiment, the p75NTR(NBP) portion of the fusion protein of the invention comprises or consists of the amino acid sequence set forth in SEQ ID NO. 1. In another preferred embodiment, the p75NTR(NBP) portion of the fusion protein of the invention comprises or consists of the amino acid sequence set forth in SEQ ID No. 2. In another preferred embodiment, the p75NTR(NBP) portion of the fusion protein of the invention comprises or consists of the amino acid sequence set forth in SEQ ID No. 3. In another preferred embodiment, the p75NTR(NBP) portion of the fusion protein of the invention comprises or consists of the amino acid sequence set forth in SEQ ID No. 4. In another preferred embodiment, the p75NTR(NBP) portion of the fusion protein of the invention comprises or consists of the amino acid sequence set forth in SEQ ID No. 5. In another preferred embodiment, the p75NTR(NBP) portion of the fusion protein of the invention comprises or consists of the amino acid sequence set forth in SEQ ID No. 6. In another preferred embodiment, the p75NTR(NBP) portion of the fusion protein of the invention comprises or consists of the amino acid sequence set forth in SEQ ID No. 7. In another preferred embodiment, the p75NTR(NBP) portion of the fusion protein of the invention comprises or consists of the amino acid sequence set forth in SEQ ID No. 8. In another preferred embodiment, the p75NTR(NBP) portion of the fusion protein of the invention comprises or consists of the amino acid sequence set forth in SEQ ID No. 9. In another preferred embodiment, the p75NTR(NBP) portion of the fusion protein of the invention comprises or consists of the amino acid sequence set forth in SEQ ID No. 10. In another preferred embodiment, the p75NTR(NBP) portion of the fusion protein of the invention comprises or consists of the amino acid sequence set forth in SEQ ID No. 11. In another preferred embodiment, the p75NTR(NBP) portion of the fusion protein of the invention comprises or consists of the amino acid sequence set forth in SEQ ID No. 12. In another preferred embodiment, the p75NTR(NBP) portion of the fusion protein of the invention comprises or consists of the amino acid sequence set forth in SEQ ID No. 13. In another preferred embodiment, the p75NTR(NBP) portion of the fusion protein of the invention comprises or consists of the amino acid sequence set forth in SEQ ID No. 14. In another preferred embodiment, the p75NTR(NBP) portion of the fusion protein of the invention comprises or consists of the amino acid sequence set forth in SEQ ID No. 15. In another preferred embodiment, the p75NTR(NBP) portion of the fusion protein of the invention comprises or consists of the amino acid sequence set forth in SEQ ID No. 16. In another preferred embodiment, the p75NTR(NBP) portion of the fusion protein of the invention comprises or consists of the amino acid sequence set forth in SEQ ID No. 17. In another preferred embodiment, the p75NTR(NBP) portion of the fusion protein of the invention comprises or consists of the amino acid sequence set forth in SEQ ID No. 18. In another preferred embodiment, the p75NTR(NBP) portion of the fusion protein of the invention comprises or consists of the amino acid sequence set forth in SEQ ID No. 19.

In another preferred embodiment, the Fc portion of the fusion protein of the invention comprises or consists of the amino acid sequence set forth in SEQ ID No. 20. In another preferred embodiment, Fc portion of the fusion protein of the invention comprises or consists of the amino acid sequence set forth in SEQ ID No. 21. In another preferred embodiment, the Fc portion of the fusion protein of the invention comprises or consists of the amino acid sequence set forth in SEQ ID No. 22. In another preferred embodiment, the Fc portion of the fusion protein of the invention comprises or consists of the amino acid sequence set forth in SEQ ID No. 23. In another preferred embodiment, the Fc portion of the fusion protein of the invention comprises or consists of the amino acid sequence set forth in SEQ ID No. 24.

Preferably the p75NTR neurotrophin binding protein, p75NTR(NBP), is pegylated, further preferably it is glycosylated.

In addition, the skilled person would understand that the therapeutic potential of the molecule of the present invention may be enhanced by the introduction of defined mutations in the crystallizable fragment (Fc) domains eg YTE (M252Y/S254T/T256E) and LS (M428L/N434S). Such techniques are well known to one skilled in the art. The effect of introducing such a mutation typically results in a molecule with increased half-life and prolonged duration of action. The effects of introducing the mutation are not limited to increased half life and duration of action.

The p75NTR(NBP)-Fc fusion protein of the present invention preferably binds to any one or more of NGF, BDNF, NT3 or NT4/5 with a binding affinity (K_{d}) of between about 0.01 nM to about 50 nM. In some preferred embodiments, the binding affinity (K_{d}) is between about 0.01 nM and any of about 0.1 nM, 0.2 nM, 0.5 nM, 1 nM, 1.5 nM, 2 nM, 2.5 nM, 3 nM, 3.5 nM, 4 nM, 4.5 nM, 5 nM, 5.5 nM, 6 nM, 6.5 nM, 7 nM, 7.5 nM, 8 nM, 8.5 nM, 9 nM, 9.5 nM, 10 nM, 15 nM, 20 nM, 25 nM, 30 nM, 35 nM, 40 nM, 45 nM or 50 nM as measured in an in vitro binding assay for NGF, BDNF, NT3 or NT4/5 such as described herein preferably as measured by surface plasmon resonance at 20° C. In some further preferred embodiments, binding affinity (K_{d}) is or is less than any of about 250 pM, 300 pM, 350 pM, 400 pM, 450 pM, 500 pM, 550 pM, 600 pM, 650 pM, 700 pM, 750 pM, 800 pM, 850 pM, 950 pM or 1 nM as measured in an in vitro binding assay for p75NTR(NBP)-Fc fusion protein with the neurotrophins such as described herein, preferably as measured by surface plasmon resonance at 20° C. In a further more preferred embodiment the binding affinity (K_{d}) is about 0.3 nM or about 1 nM, as measured in an in vitro binding assay for p75NTR(NBP)-Fc fusion protein with the neurotrophins such as described herein, preferably as measured by surface plasmon resonance at 20° C.

Preferably the p75NTR(NBP)-Fc fusion protein of the invention is for use in the treatment of pain. Without wishing to be bound by any particular theory, the inventors believe that the p75NTR(NBP)-Fc fusion protein achieves efficacy in the treatment of pain by effecting the functional activity of the aforementioned neurotrophins, (defined as modulating or up or down regulating the functional activity of the neurotrophins) NGF, BDNF, NT3 or NT4/5, for example the functional activity of the aforementioned neurotrophins resulting from their interaction with their respective receptors.

Preferably the p75NTR(NBP)-Fc fusion protein effects the functional activity of BDNF as assessed by functional assay of any of growth and differentiation of neurons and synapses, survival and differentiation in neuronal cell culture, Trk signalling, stimulation of axon outgrowth in vitro or in vivo.

Preferably the p75NTR(NBP)-Fc fusion protein effects the functional activity of NGF as assessed by measuring NGF binding to and activation of TrkA, as demonstrated in classical neuron survival assays (such as provided in Cowan et al. Annu. Rev. Neurosci. 2001; 24:551-600).

Preferably the p75NTR(NBP)-Fc fusion protein effects the functional activity of NT3 as assessed by measuring NT3 binding to and activation of endogenous Trk receptor activity, as demonstrated in Trk receptor phosphorylation, mitogen-activated protein kinase phosphorylation reporter assays or cell survival and neurite extension assays.

Preferably the p75NTR(NBP)-Fc fusion protein effects the functional activity of NT4/5 as assessed by measuring NT4/5 in vitro or in vivo phosphorylation and activation assays for example in myelin basic protein (MBP) phosphorylation assays or alternatively in vivo in a Matrigel angiogenesis assay of vascular endothelial growth factor (VEGF)/basic fibroblast growth factor-induced angiogenesis.

Preferably the p75NTR(NBP)-Fc fusion protein binds to the contact residues of one or more of the neurotrophins NGF, NT3, BDNF and NT4/5 as shown in He and Garcia (2001) Science, 301, pages 870-805.

Preferably the p75NTR(NBP)-Fc fusion protein is soluble, preferably soluble in aqueous solution, preferably soluble in a biological fluid such as serum, plasma, blood.

As used herein, the term, “Fc” or “immunoglobulin Fc” or “Ig Fc” is understood to mean the carboxyl-terminal portion of an immunoglobulin chain constant region, preferably an immunoglobulin heavy chain constant region, or a portion thereof. Preferably the immunoglobulin Fc comprises 1) a CH1 domain, a CH2 domain, and a CH3 domain, optionally with an immunoglobulin hinge region, 2) a CH1 domain and a CH2 domain, optionally with an immunoglobulin hinge region, 3) a CH1 domain and a CH3 domain, optionally with an immunoglobulin hinge region, 4) a CH2 domain and a CH3 domain, optionally with an immunoglobulin hinge region or 5) a combination of two or more domains selected from but not limited to CH1, CH2 and CH3 optionally combined with an immunoglobulin hinge region.

According to the present invention, the p75NTR(NBP)-Fc fusion protein preferably demonstrates advantageous biological properties of improved solubility of p75NTR(NBP) and/or stability of p75NTR(NBP) and/or improved serum half life p75NTR(NBP). Improved solubility is desirable in order that bioavailability of the p75NTR(NBP) is maximized on administration and accurate dosage of the p75NTR(NBP) can be determined and carried out. Improved solubility is advantageous to overcome the problem of aggregates which are undesirable causing pain in delivery in-vivo and leading to potential inflammation. Improved serum half life has the advantage of facilitating reduced levels or reduced frequency of dose requirement during use for treatment in order to achieve the equivalent or maintained therapeutic effect of the p75NTR(NBP) delivered. A prolonged half life and higher stability in blood or serum has the advantage of permitting a dosage regime of less frequent dosing and/or lower dosing levels hence reducing potential toxicity or side effects in-vivo. In this case the p75NTR(NBP)-Fc fusion protein is more potent in its therapeutic effect and/or more stable in the circulation. The resulting lower or less frequent doses are advantageous in minimising any potential toxic effects or side effects potentially associated with p75NTR(NBP) administration. The molecular weight of the p75NTR(NBP)-Fc fusion protein is also increased over p75NTR(NBP) alone, this has the advantage that the molecule will be well retained in the blood circulation when administered intravenously reducing the risk of penetration to undesired sites for example the central nervous system and making the molecule suitable for retention or concentration in the tissues targeted.

Preferably the p75NTR(NBP)-Fc fusion protein demonstrates improved solubility of p75NTR(NBP) and/or improved stability of p75NTR(NBP) and/or improved serum half life in comparison to p75NTR(NBP) alone. Preferably the improved solubility is solubility in an aqueous solution such as water preferably with excipients such as buffers and/or salts at preferably at a physiological pH, preferably at between pH 5 to pH 8, preferably about pH 7, or is solubility in a biological fluid such as serum or blood. Preferably the improved stability is stability of activity or structural integrity of the p75NTR(NBP) protein due to the effects of denaturation, oxidation, fragmentation or aggregation over a period of time, during a period storage or following freeze and thaw. Structural stability can be judged by standard measures of denaturation, oxidation, aggregation or aggregation, stability of activity can be measured by the binding or functional assays disclosed herein, methods of measuring protein serum half life are known.

Preferably the p75NTR(NBP)-Fc fusion protein can be expressed at high levels from variety of mammalian host cells to provide a single species and can be efficiently purified by affinity chromatography for example by binding to Staphylococcus aureus protein A. Preferably the p75NTR(NBP)-Fc fusion protein can dimerise and preferably the dimer has increased affinity to neurotrophins NGF, BDNF, NT3 or NT4/5 in comparison to p75NTR(NBP) alone. Tighter binding has the advantage of higher potency and a higher therapeutic efficacy as judged by the p75NTR(NBP) effects for example as determined by neurotrophin functional assays disclosed herein. Higher potency has the benefit that the p75NTR(NBP)-Fc fusion protein can be used at lower dosage amounts to achieve the same therapeutic efficacy hence reducing potential toxicity or side effects in-vivo.

Preferably the p75NTR(NBP)-Fc fusion protein of the invention has a half life in-vivo of about or more than any one of 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152,154, 156, 158, 160, 62, 164, 166, 168, 170, 172, 174, 176, 178, 180, 182, 184, 186, 188, 190, 192, 194, 196, 198, 200, 202, 204, 206, 208 or 210 hours+/−1 hour, further preferably the p75NTR(NBP)-Fc fusion protein of the invention has a half life in-vivo of about or more than 24 hours.

Further preferably the p75NTR(NBP)-Fc fusion protein of the invention has a half life in-vitro of about or more than any one of 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152,154, 156, 158, 160, 62, 164, 166, 168, 170, 172, 174, 176, 178, 180, 182, 184, 186, 188, 190, 192, 194, 196, 198, 200, 202, 204, 206, 208 or 210 days+/−1 day, further preferably the p75NTR(NBP)-Fc fusion protein of the invention has a half life in-vitro of about or more than 6 days. Preferably the stability is measured at about physiological pH, in a buffered aqueous solution, preferably at 20° C. or 37° C.

According to the foregoing preferred embodiments, preferably the in-vivo half life is half life in rat or half life in human, more preferably in human. Preferably the half life is determined from serum measurements of the levels of p75NTR(NBP)-Fc fusion protein of the invention following administration in-vivo for example by intravenous or subcutaneous injection.

The p75NTR(NBP) and immunoglobin Fc portions of the p75NTR(NBP)-Fc fusion protein are connected by a linker. The linker preferably the linker comprises or consists of one or a plurality of amino acids or comprises or consists of a polypeptide sequence of amino acids, preferably about 1 to about 25 amino acids, preferably any one of 1, 2, 3, 4, 5, 6, 7, 8, or 9 amino acids further preferably any one of about 10, 11, 12, 13, 14, 15, 16, 17, 18, 19, 20, 21 22, 23 or 24 amino acids, most preferably 13 amino acids.

Preferably the linker comprises or consists of a polypeptide sequence of amino acids that lacks any stable secondary structure such as alpha helix, beta strand, 3₁₀ helix and pi helix, polyproline helix, alpha sheet. Preferably the linker region comprises or consists of a polypeptide sequence of amino acids that defines a flexible or dynamic or unstructured polypeptide such as for example a flexible loop, random coil or flexible turn, such unstructured polypeptides are often found connecting regions of secondary structure in large protein molecules.

Preferably the linker is a polypeptide sequence of amino acids that comprises greater than or about 50% glycine and/or alanine and/or serine in p75NTR(NBP), further preferably greater than or about 55%, 60%, 65%, 70%, 75%, 80%, 85%, 90%, 95% or 100% glycine and/or alanine and/or serine in p75NTR(NBP). Preferably the linker region comprises or consists of a polypeptide sequence of amino acids that comprises both glycine and serine, preferably with a greater proportion of glycine that serine, preferably the linker region comprises or consists of flexible linkers.

Without wishing to be bound by any particular theory, the inventors believe that flexible linkers overcome or prevent steric hindrance which could interfere with the aforementioned neurotrophin binding ability or biological activity of the p75NTR(NBP)-Fc fusion when compared to p75NTR(NBP) alone. Hence the linker region preferably permits flexibility between the p75NTR(NBP) portion and the immunoglobin Fc portion and allows retention of or improvement of the aforementioned biological activity of p75NTR(NBP)-Fc fusion protein in comparison to free or native p75NTR(NBP) alone as determined by binding to neurotrophins using binding assays such as described herein.

Further preferably the linker is immunologically inert, such that it does not trigger complement mediated lysis, does not stimulate antibody-dependent cell mediated cytotoxicity (ADCC), does not activate microglia or T-cells. Preferably the linker region is reduced in one or more of these activities.

Further preferably the linker comprises or consists of a polypeptide known or predicted from structural analysis or structural prediction to be a flexible or dynamic or unstructured polypeptide or to lack a stable secondary structure.

The linker comprises a peptide of formula Gₓ, where x is 1, 2, 3, 4, 5 or 6

In a preferred embodiment the linker does not comprise or consist of the sequence GGGGS.

In a further embodiment the linker is GGG.

The p75NTR(NBP)-Fc fusion protein of the invention may also comprise a proteolytic cleavage site, optionally interposed between the p75NTR(NBP) portion and the immunoglobin Fc portion. The proteolytic cleavage site may be located in the linker or at the junction of the linker with either the p75NTR(NBP) portion or/and the immunoglobin Fc portion. The p75NTR(NBP) may optionally be cleaved from the immunoglobin Fc portion prior to formulation and or administration for therapeutic purposes.

Preferably the linker and/or the immunoglobin Fc portion do not impair or significantly impair the p75NTR(NBP) portion:
(a) effect on the functional activity of the neurotrophins (defined as modulating or up or down regulating the functional activity of the neurotrophins) NGF, BDNF, NT3 or NT4/5,
(b) binding affinity for any of NGF, BDNF, NT3 or NT4/5 with a binding affinity of between about 0.1 nM to about 50 nM
(c) ability to binds to each of the neurotrophins NGF, NT3, BDNF and NT4/5, preferably human NGF, NT3, BDNF and NT4/5.

According to another aspect of the invention there is provided a nucleic acid molecule encoding the p75NTR(NBP)-Fc fusion protein according to the first or second aspects. Preferably the nucleic acid molecule is for use in the treatment of pain.

According to a preferred embodiment of the present invention the nucleic acid molecule may further comprise a region encoding a signal sequence, preferably a p75NTR signal sequence for example a DNA or RNA sequence.

According to another aspect of the invention there is provided a replicable expression vector for transfecting a cell, the vector comprising the nucleic acid molecule of the third aspect, preferably the vector is a viral vector. Preferably the vector is for use in the treatment of pain.

Further according to the above aspects of the invention there is provided a method of expressing the nucleic acid molecule or the vector of the invention to produce or secrete the p75NTR(NBP)-Fc fusion protein. Preferably the method comprises the introduction of the nucleic acid molecule or vector into a cell and expression of the nucleic acid therein to produce or secrete the p75NTR(NBP)-Fc fusion protein. Preferably the nucleic acid molecule or vector is introduced into the cell in-vitro alternatively in-vivo. Preferably the expressed p75NTR(NBP)-Fc fusion protein is expressed in-vitro, optionally further isolated and purified, alternatively preferably the expressed p75NTR(NBP)-Fc fusion protein is expressed in-vivo, preferably the in-vivo expression constitutes gene therapy. Preferably the vector is a replicable expression vector, optionally for transfecting a mammalian cell, preferably the vector is a viral vector.

According to another aspect of the invention there is provided a host cell harbouring the nucleic acid molecule or vector of either the third or fourth aspect, preferably the cell is a mammalian cell.

According to another aspect of the invention there is provided the p75NTR(NBP)-Fc fusion protein for use in the treatment of pain, or a nucleic acid or vector for use in the treatment of pain. Pain may include but is not limited to:
(a) acute pain and/or spontaneous pain,
(b) chronic pain and or on-going pain,
(c) inflammatory pain including any one of arthritic pain, pain resulting from osteoarthritis or rheumatoid arthritis, resulting from inflammatory bowel diseases, psoriasis and eczema
(d) nociceptive pain,
(e) neuropathic pain, including painful diabetic neuropathy or pain associated with post-herpetic neuralgia,
(f) hyperalgesia,
(g) allodynia,
(h) central pain, central post-stroke pain, pain resulting from multiple sclerosis, pain resulting from spinal cord injury, or pain resulting from Parkinson's disease or epilepsy,
(i) cancer pain,
(j) post-operative pain,
(k) visceral pain, including digestive visceral pain and non-digestive visceral pain, pain due to gastrointestinal (GI) disorders, pain resulting from functional bowel disorders (FBD), pain resulting from inflammatory bowel diseases (IBD), pain resulting from dysmenorrhea, pelvic pain, cystitis, interstitial cystitis or pancreatitis,
(l) musculo-skeletal pain, myalgia, fibromyalgia, spondylitis, sero-negative (non-rheumatoid) arthropathies, non-articular rheumatism, dystrophinopathy, Glycogenolysis, polymyositis, pyomyositis,
(m) heart or vascular pain, pain due to angina, myocardical infarction, mitral stenosis, pericarditis, Raynaud's phenomenon, scleredoma, scleredoma or skeletal muscle ischemia,
(n) head pain including migraine, migraine with aura, migraine without aura cluster headache, tension-type headache.
(o) orofacial pain, including dental pain, temporomandibular myofascial pain or tinnitus, or
(p) back pain, bursitis, menstrual pain, migraine, referred pain, trigeminal neuralgia, hypersensitisation, pain resulting from spinal trauma and/or degeneration or stroke.

Treatment of pain includes, but is not limited to, preventing, ameliorating, controlling, reducing incidence of, or delaying the development or progression of pain.

According to another aspect of the invention there is provided the p75NTR(NBP)-Fc fusion protein according to the first or second aspect or the preferred embodiments thereof, or the nucleic acid molecule or vector according to the third and fourth aspects wherein the p75NTR(NBP)-Fc fusion protein or the nucleic acid molecule or vector is for separate, sequential or simultaneous use in a combination combined with a second pharmacologically active compound. Preferably the second pharmacologically active compound of the combination may include but is not limited to;
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflunisal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;
a barbiturate sedative, e.g. amobarbital, aprobarbital, butabarbital, butalbital, mephobarbital, metharbital, methohexital, pentobarbital, phenobarbital, secobarbital, talbutal, theamylal or thiopental;
a benzodiazepine having a sedative action, e.g. chlordiazepoxide, clorazepate, diazepam, flurazepam, lorazepam, oxazepam, temazepam or triazolam;
an H₁ antagonist having a sedative action, e.g. diphenhydramine, pyrilamine, promethazine, chlorpheniramine or chlorocyclizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphenadrine;
an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®, a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
an alpha-adrenergic, e.g. doxazosin, tamsulosin, clonidine, guanfacine, dexmedetomidine, modafinil, or 4-amino-6,7-dimethoxy-2-(5-methane-sulfonamido-1,2,3,4-tetrahydroisoquinol-2-yl)-5-(2-pyridyl) quinazoline;
a tricyclic antidepressant, e.g. desipramine, imipramine, amitriptyline or nortriptyline;
an anticonvulsant, e.g. carbamazepine, lamotrigine, topiramate or valproate;
a tachykinin (NK) antagonist, particularly an NK-3, NK-2 or NK-1 antagonist, e.g. (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
a muscarinic antagonist, e.g oxybutynin, tolterodine, propiverine, trospium chloride, darifenacin, solifenacin, temiverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, belaperidone, palindore, eplivanserin, osanetant, rimonabant, meclinertant, Miraxion® or sarizotan;
a vanilloid receptor agonist (e.g. resiniferatoxin) or antagonist (e.g. capsazepine);
a beta-adrenergic such as propranolol;
a local anaesthetic such as mexiletine;
a corticosteroid such as dexamethasone;
a 5-HT receptor agonist or antagonist, particularly a 5-HT_{1B/1D} agonist such as eletriptan, sumatriptan, naratriptan, zolmitriptan or rizatriptan;
a 5-HT_{2A} receptor antagonist such as R(+)-alpha-(2,3-dimethoxy-phenyl)-1-[2-(4-fluorophenylethyl)]-4-piperidinemethanol (MDL-100907);
a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
Tramadol®;
a PDEV inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
a cannabinoid;
metabotropic glutamate subtype 1 receptor (mGluR1) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
a noradrenaline (norepinephrine) reuptake inhibitor, such as maprotiline, lofepramine, mirtazapine, oxaprotiline, fezolamine, tomoxetine, mianserin, buproprion, buproprion metabolite hydroxybupropion, nomifensine and viloxazine (Vivalan®), especially a selective noradrenaline reuptake inhibitor such as reboxetine, in particular (S,S)-reboxetine;
a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;
an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-5-chloro-3-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl) butyl]thio]-6-(trifluoromethyl)-3 pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;
an acetylcholinesterase inhibitor such as donepezil;
a prostaglandin E₂ subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1S)-1-({[5-chloro-2-(3-fluorophenoxy)pyridin-3-yl]carbonyl}amino)ethyl]benzoic acid;
a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870,
a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl), 1,4-benzoquinone (CV-6504);
a sodium channel blocker, such as lidocaine; or
a 5-HT3 antagonist, such as ondansetron;
   and the pharmaceutically acceptable salts and solvates thereof.

According to a further aspect of the present invention there is provided a method of treating, preventing, ameliorating, controlling, reducing incidence of, or delaying the development or progression of pain or any of the foregoing pain in an individual, comprising administration to the individual of an effective amount of the p75NTR(NBP)-Fc fusion protein according to the first or second aspect or the preferred embodiments thereof, or the nucleic acid molecule or vector according to the third and fourth aspects.

In another preferred embodiment, the p75NTR(NBP)-FC fusion protein of the present invention are suitable for the treatment of osteoarthritis. In particular for arresting, slowing and reversing progression of the disease. In a particular embodiment the proteins of the invention provide a cure for osteoarthritis.

In one embodiment of the invention it is suitable for treatment of osteoarthritis in humans

In one embodiment of the invention it is suitable for treatment of osteoarthritis in veterinary medical fields. Preferably the individual is a mammal, for example a companion animal such as a horse, cat or dog or a farm animal such as a sheep, cow or pig. Most preferably the animal is a dog.

According to an eighth aspect of the present invention there is provided a pharmaceutical composition for any one or more of treating, preventing, ameliorating, controlling, reducing incidence of, or delaying the development or progression of pain or any of the foregoing pain, comprising the p75NTR(NBP)-Fc fusion protein according to the first or second aspect or the preferred embodiments thereof, or the nucleic acid molecule or vector according to the third and fourth aspects and a pharmaceutically acceptable carrier and/or an excipient.

Preferably the p75NTR(NBP)-Fc fusion protein according to the first or second aspects or the preferred embodiments thereof, or the nucleic acid molecule or vector according to the third and fourth aspects or the pharmaceutical of the eighth aspect is prepared for or suitable for oral, sublingual, buccal, topical, rectal, inhalation, transdermal, subcutaneous, intravenous, intra-arterial, intramuscular, intracardiac, intraosseous, intradermal, intraperitoneal, transmucosal, vaginal, intravitreal, intra-articular, peri-articular, local or epicutaneous administration.

Preferably the p75NTR(NBP)-Fc fusion protein according to the first or second aspect or the preferred embodiments thereof, or the nucleic acid molecule or vector according to the third and fourth aspects or the pharmaceutical composition of the eighth aspect is prepared for or suitable for administration prior to and/or during and/or after the onset of pain or for such use.

Preferably the p75NTR(NBP)-Fc according to the first or second aspect or the preferred embodiments thereof, or the nucleic acid molecule or vector according to the third and fourth aspects or the pharmaceutical composition of the eighth aspect is for or prepared for administration between once to 7 times per week, further preferably between once to four times per month, further preferably between once to six times per 6 month period, further preferably once to twelve times per year. Preferably the medicament is to be or prepared to be peripherally administered in a period including but not limited to: once daily, once every two, three, four, five or six days, weekly, once every two weeks, once every three weeks, monthly, once every two months, once every three months, once every four months, once every five months, once every six months, once every seven months, once every eight months, once every nine months, once every ten months, once every eleven months or yearly.

Further preferably the p75NTR(NBP)-Fc fusion protein according to the first or second aspect or the preferred embodiments thereof, or the nucleic acid molecule or vector according to the third and fourth aspects or the pharmaceutical composition of the eighth aspect is to be or prepared to be peripherally administered via a route including but not limited to one or more of; orally, sublingually, buccally, topically, rectally, via inhalation, transdermally, subcutaneously, intravenously, intra-arterially or intramuscularly, via intracardiac administration, intraosseously, intradermally, intraperitoneally, transmucosally, vaginally, intravitreally, epicutaneously, intra-articularly, peri-articularly or locally.

Preferably the p75NTR(NBP)-Fc fusion protein according to the first or second aspect or the preferred embodiments thereof, or the nucleic acid molecule or vector according to the third and fourth aspects or the pharmaceutical composition of the eighth aspect is for or is prepared for administration at a concentration of between about 0.05 to about 200 mg/ml; preferably at any one of about 0.05, 0.1, 0.5, 1, 5, 10, 15, 20, 25, 30, 35, 40, 45, 50, 55, 60, 65, 70, 75, 80, 85, 90, 95, 100, 110, 120, 130, 140, 150, 160, 170, 180, 190 or 200 mg/ml+/−about 10% error, most preferably at about 3 mg/ml in veterinary applications and 0.1 in humans.

Preferably the p75NTR(NBP)-Fc fusion protein according to the first or second aspect or the preferred embodiments thereof, or the nucleic acid molecule or vector according to the third and fourth aspects or the pharmaceutical composition of the eighth aspect is for or is prepared for administration at a concentration of between about 0.1 to about 200 mg/kg of body weight; preferably at any one of about 0.5, 1, 5, 10, 15 20, 25, 30, 35, 40, 45, 50, 55, 60, 65, 70, 75, 80, 85, 90, 95, 100, 110, 120, 130, 140, 150, 160, 170, 180, 190 or about 200 mg/kg of body weight+/−about 10% error, most preferably at about 10 mg/kg in veterinary applications and 0.3 in humans

According to a ninth aspect of the present invention there is provided a kit comprising:
(a) the p75NTR(NBP)-Fc fusion protein according to the first or second aspect or the preferred embodiments thereof, or the nucleic acid molecule or vector according to the third and fourth aspects or the pharmaceutical composition of the eighth aspect; and
(b) instructions for the administration of an effective amount of said p75NTR(NBP)-Fc fusion protein, nucleic acid molecule, vector or pharmaceutical composition to an individual for any one or more of the prevention or treatment of pain or for ameliorating, controlling, reducing incidence of, or delaying the development or progression of pain.

The kit may include one or more containers containing the p75NTR(NBP)-Fc fusion protein, nucleic acid, vector or pharmaceutical composition described herein and instructions for use in accordance with any of the methods and uses of the invention. The kit may further comprise a description of selecting an individual suitable for treatment based on identifying whether that individual has a pain or a symptom of pain or is at risk of having such. The instructions for the administration of the pharmaceutical composition may include information as to dosage, dosing schedule and routes of administration for the intended treatment.

According to yet another aspect of the present invention there is provided the p75NTR(NBP)-Fc fusion protein according to the first or second aspect or the preferred embodiments thereof, or the nucleic acid molecule or vector according to the third and fourth aspects or the pharmaceutical composition of the eighth aspect for use in any one or more of the prevention or treatment or for ameliorating, controlling, reducing incidence of, or delaying the development or progression of a condition or the symptoms of a condition associated with any one or more of the neurotrophins NGF, BDNF, NT-3, NT-4/5.
NGF (Nerve growth factor) binds with at least two classes of receptors: the p75NTR and TrkA, a transmembrane tyrosine kinase, it is involved in axonal growth, branching and elongation. Conditions and symptoms associated with NGF are known. NGF is expressed in and associated with inflammatory conditions and pain [Protein Sequence NP_002497.2, NP_038637]. Also, NGF has been shown to play a role in number cardiovascular diseases, such as coronary atherosclerosis, obesity, type 2 diabetes, and metabolic syndrome as well as in Multiple Sclerosis. Reduced plasma levels of NGF (and also of BDNF) have been associated with acute coronary syndromes and metabolic syndromes. NGF is also related to various psychiatric disorders, such as dementia, depression, schizophrenia, autism, Rett syndrome, anorexia nervosa, and bulimia nervosa and has also been implicated in development of Alzheimer's disease and neurodegenerative disorders. NGF has also been shown to accelerate wound healing and there is evidence that it could be useful in the treatment of skin ulcers and corneal ulcers, it has been shown to reduce neural degeneration and to promote peripheral nerve regeneration in rats.
BDNF (brain-derived neurotrophic factor) is a neurotrophin which supports neuronal survival and growth during development of the nervous system [Protein Sequence NP_001137277.1, NP_001041604]. BDNF binds cell surface receptors TrkB and p75NTR and also modulates the activity of Alpha-7 nicotinic receptor. Conditions and symptoms associated with BDNF are known. BDNF has been shown to play a significant role in the transmission of physiologic and pathologic pain, particularly in models of acute pain, inflammatory pain and neuropathic pain, where BDNF synthesis is found to be greatly increased; also BDNF has been shown to be up-regulated in conditions of chronic pain as well as further conditions such as eczema and psoriasis. Down-regulation of BDNF is seen in depression, schizophrenia, obsessive-compulsive disorder, Alzheimer's disease, Huntington's disease, Rett syndrome, and dementia, as well as anorexia nervosa and bulimia nervosa.
Neurotrophin-4 (NT-4), also known as neurotrophin-5 (NT-5), is a neurotrophic factor that signals predominantly through the p75NTR and TrkB receptors and promotes the survival of peripheral sensory sympathetic neurons. The mature peptide of this protein is identical in all mammals examined including human, pig, rat and mouse. [Protein Sequence NP_006170, NP_937833]. NT-4 is synthesized by most neurons of the dorsal root ganglion (DRG) and those in the paravertebral and prevertebral sympathetic ganglia, spinal dorsal and ventral horn and is found expressed in many tissues including the prostate, thymus, placenta and skeletal muscle. Conditions and symptoms associated with NT-4/5 are known. Defects in NT4/5 are associated with susceptibility to primary open angle glaucoma. Neurotrophin 4 has also been shown to contribute to breast cancer cell survival and is a target to inhibit tumour growth. NT-4/5 is known to be involved in pain-signalling systems such as nociceptive pain, upregulation of NT-4/5 is also seen in chronic inflammatory conditions of the skin, such as dermatitis, eczema, prurigo lesions of atopic dermatitis. Down regulation of NT-4/5 is seen in Alzheimer's Disease, Huntington's disease.
Neurotrophin-3 (NT-3), is a neurotrophin that is structurally related to beta-NGF, BDNF, and NT-4, and that controls survival and differentiation of mammalian neurons and the maintenance of the adult nervous system, and may affect development of neurons in the embryo when it is expressed in human placenta. Conditions and symptoms associated with NT3 are known. NTF3-deficient mice generated by gene targeting display severe movement defects of the limbs. NT-3 signals through the Trk receptors and promotes the growth and survival of nerve and glial cells [Protein Sequence NP_001096124.1 and NP_032768]. The amino acid sequences of human, Mouse and rat NT-3 are identical. NT3 and its cognate receptor, tyrosine kinase C (TrkC), are known to modulate neuropathic pain and nociceptive pain and the mechanism of nociception and proprioception, for example NT3 expression is increased in the small DRG cells of neuropathic animals. NT3 expression is also associated with neuropathies such as diabetic polyneuropathy and HIV-related neuropathy, large fiber neuropathy including atrophy, it is further involved in the development of hyperalgesia (a decrease in the threshold of a normally noxious stimuli), allodynia (a non-noxious stimulus becomes noxious), and spontaneous pain (pain in the apparent absence stimuli) and is a known modulator of muscle pain.

The invention will now be described by reference to the following examples which are provided to illustrate, but not to limit, the invention.

### EXAMPLES

p75-NTR Fc-Fusion Protein Design

The specific allotype of the Fc portion of the p75-NTR Fc-fusion protein was the IgG pCon vector IgGza (see above).

The design of a p75-NTR Fc-fusion protein proceeded in several stages:
The exact construct of the p75-NTR sequence to be used in the Fc-fusion protein was defined. Several factors were considered including:
   The p75-NTR Fc-fusion should be able to bind several neurotrophins including at least NGF, BDNF, NT-3 and NT-4; flexibility must be retained in the p75-NTR Fc-fusion protein.
   Unwanted alpha-secretase cleavage sites is present in the extracellular domain on the p75-NTR-Fc (SEQ ID No. 1), these must be removed from the sequence as they will be subjected to cleavage and consequently reduce the biological activity and PK profile of the p75-Fc product in vivo.

Appropriate empirical linkers, suitable for use to join the extracellular p75-NTR domain and the Fc in a p75-NTR Fc-fusion protein, were identified. Linker sequences containing sites that can potentially participate in Post-translational Modification (PTMs) were excluded.

Several variants of the p75-NTR Fc-fusion protein were constructed in silico using the defined p75-NTR construct with the appropriate portion of the Fc region using different potential linker sequences. Structural modelling and analysis of the C-terminus of p75-NTR extracellular domain, Fc hinge region and potential linker was attempted.

### Structural Modelling

Structural models of the proposed p75-NTR Fc-fusion protein, were generated using Lonza's modelling platform. Candidate structural template fragments for the p75-NTR and the Fc portion were scored, ranked and selected from both an in-house antibody database base and the Protein Data Bank (PDB), on their sequence identity, as well as qualitative crystallographic measures of the template structure, such as the resolution (in Ångström (Å)).

A sequence alignment of the structural template fragments to the p75-NTR Fc-fusion protein was generated. The template fragments along with the sequence alignment were processed by MODELLER (Sali et al. 1993 J. Mol. Biol 234, 779-815). This protocol creates conformational restraints derived from the set of aligned structural templates. An ensemble of structures that satisfy the restraints is created by conjugate gradient and simulated annealing optimization procedures. One or more model structures are selected from this ensemble on the basis of an energy score, derived from the score of the protein structure and satisfaction of the conformational restraints. The models were inspected and the side chains of the positions which differ between the target and template were optimized using a side chain optimization algorithm and energy minimized. A suite of visualization and computational tools were used to assess the conformational variability of the structures, as well as the core and local packing of the domains to select one or more preferred models.

p75-NTR Fc-Fusion Protein Design

The key requirements of the linkers chosen for the variants are to allow the flexibility of the fusion partner in the Fc-fusion protein, to avoid introducing any residues capable of bearing PTMs and to maintain a low immunogenicity risk.

Turning to the process by which the protein is expressed, methods for expressing proteins are well known to those skilled in the art.

The process started with codon optimisation of the DNA sequence to allow efficient expression in CHO cell lines with a signal sequence at the N-terminus that directs the protein for secreted expression and appropriate restriction enzyme sites to enable cloning into cloning into the glutamine synthetase (GS) vectors and a Kozak sequence between the 5′ restriction enzyme site and the ‘ATG’ start codon. These sequences were submitted to and synthesised by Life Technologies and provided within a cloning vector. Other vectors with different secretion sequences and a different selectable marker (example dihydrofolate reductase (DHFR) instead of GS). The 1405 base pair (bp) DNA fragment encoding the protein was removed from the cloning vector into the GS expression vector pXC-17 via HindIII and EcoR1 digestion. The digested products and vector were ligated and the ligated products used to transform chemically competent Escherichia coli (E. coli) cells (for example TOP10 cells). Single colonies were analysed for the correct insert and one positive clone was selected and plasmid DNA prepared and sequence (in forward reverse orientation) to ensure the correct sequence was present. Sufficient linearised DNA for the cell line construction process was generated by linearisation of the plasmid with the restriction enzyme PvuI.

Lonza Biologics' mammalian Chinese Hamster Ovary (CHO) K1SV Glutamine Synthetase-Knock Out (GS-KO) expression system was used to produce the protein. The CHOK1SV GS-KO host cell line is a derivative of the CHOK1SV host cell line with the endogenous gene for GS ‘knocked out’. The host cell line was derived from Lonza Biologics' CHOK1SV GS-KO host working cell bank.

Affinity of Sequence 1-19 (SEQ ID No. 1-19) p75NTR-Fc for NGF

A Biacore chip was prepared in an experiment in which Protein A was amine coupled to flow cells 1 and 2. Single cycle kinetics of NGF binding to captured p75-Fc were measured.

The binding capacity (Rₘₐₓ) of a chip surface depends of the immobilised level of the ligand (fusion protein). For a kinetics study an Rₘₐₓ of 50-100 RU is advised. By using the molecular weights of the p75-Fc and NGF, a desired immobilisation level for the fusion protein can be calculated.

Rₘₐₓ=(NGF molecular weight/fusion protein molecular weight)×immobilisation level×stoichiometric ratio:50=(13,500/102,000)×immobilisation level×1.

Hence, the immobilisation level required=(102,000/13,500)×50=378 RU Sequence 1 (SEQ ID No. 1) and Sequence 3 (SEQ ID No. 3) p75NTR-Fc and NTR-Fc were immobilised onto the Protein A chip prior to single cycle kinetics.

Using a manual run, p75-Fc was captured onto flow cell 2 of the Protein A chip until the desired level of approx. 380 RU was achieved. This was performed with a 22 second injection at a flow rate of 10 μl/min and p75-Fc concentration 10 μg/ml which resulted in 418 RU of the fusion protein captured onto the protein A surface.

In the first instance NGF concentrations of 10, 5 2.5, 1.25 and 0.625 nM were tested. These concentrations were tested as the K_{D} for the fusion protein was approximated to be within this range of NGF concentrations.

The single cycle kinetics method involved:
injecting 0.625 nM of NGF onto the captured p75-Fc for 120 seconds at 30 μl/min
this process was then repeated with an injection of NGF at 1.25 nM, followed by 2.5, 5 and 10 nM
after the final concentration of NGF had been injected a 600 second dissociation phase was performed by flowing the running buffer (HBS-EP) over the chip.

Once completed the chip was regenerated back to its Protein A surface by injecting 10 mM Glycine HCl, pH 2 for 60 seconds at 30 μl/min.

p75-Fc was then captured onto the chip by performing a 38 second injection at a flow rate of 10 μl/min at a concentration of 10 μg/ml. This achieved the desired level of 430 RU. The single cycle kinetics procedure described above was then repeated.

### Data Analysis

The fusion protein-NGF binding data was analysed in the following manner using the Biacore T200 evaluation software v1:
Data is recorded for the binding of NGF to the fusion protein on flow cell 2 (Fc=2) and for NGF flowing over the control flow cell 1 (Fc=1; protein A alone).
The data from Fc=1 is then subtracted from Fc=2 to give “2-1” binding data.
The 2-1 binding data for an injection of 0 nM (HBS-EP running buffer alone) is then subtracted from all the 2-1 binding data to control for any drifts in baseline throughout the experiment.
Finally, this data is then fitted to a 1:1 binding model to calculate binding characteristics including association rates (ka), dissociation rates (kd) and affinities (K_{D}).

The sequences showed suitable affinity for NGF.

P75NTR-Fc is analgesic.

The aim of this study was to investigate the effects of chronic exposure of p75NTR-Fc on pain efficacy in monosodium-iodoacetate (MIA) induced osteoarthritis (OA) in rats.

Previously, we also have shown that an assessment of spontaneous pain could made by measurement of static weight bearing using an incapacitance tester and that this correlated with the histopathology of the knee. Pre-clinical studies using novel therapies for pain have been criticized for their capability to induce bias in the data. To address this, both the left and right knees were randomly chosen for the induction of OA, and all operators of the everyday in vivo tasks were blinded to the status of each knee. Typically from the literature induction of OA is carried out in the right knee only, but in a previous studies we found no consistent differences between the induction of OA in the left versus the right knee regardless of the time point or dose of MIA used.

### Preparation of MIA

MIA was prepared at 0.3 mg/50 μl ETF-PBS (the volume used for each intra-articular injection) which is equivalent to 6 mg/ml stock solution. 302 mg of MIA was weighed out and dissolved in 50.3 ml ETF-PBS. The MIA was prepared a day in advance and was stored at 4° C. in the dark until required.

### Animals

185 male Wistar rats (from Charles River UK) weighing 110-130 g on arrival were used in this study. Each animal was checked on arrival and appeared outwardly healthy. They were randomly assigned to a cage of two and each rat was allocated a unique identification number by a tattoo on the tail. Animals were acclimatised to the animal unit for at least 10 days prior to the start of the study on day 0. Once the rats had acclimatised to their environment they were transferred to a stock/procedure room, where all the in vivo procedures were carried out. Animals were kept illuminated by fluorescent lights set to give a 12 hour light-dark cycle (on 07.00 off 19.00) as recommended in the Home Office Animals (Scientific Procedures) Act 1986. The rooms were air-conditioned and the air temperature (21° C.+/−2° C.) and relative humidity were routinely measured.

Rats were fed an irradiated diet (Scientific Animal Food and Engineering, Augy, France) and autoclaved water was available ad libitum. Each batch of diet was checked and screened routinely for composition and contaminants. Nesting and cages were autoclaved and each cage was individually ventilated (IVC system).

### Experimental Design

The study design was such that there were 21 groups of animals: control human antibody (n=6), 19 groups of tested p75NTR-Fc at 3 mg/kg p75NTR-Fc (seq ID 1-19 each with a GGG linker attached to Seq ID no 20 as Fc region) and 3 mg/kg PG-007 (biosimilar anti-NGF antibody of the Pfizer Tanezumab).

Antibodies and p75NTR-Fc were administered by subcutaneous injection every 5 days for 25 days.

Body weight was measured and a baseline blood sample was taken from the tail vein in the morning of day −2. At approximately the same time on day −1 baseline static weight bearing was measured. On day 0, again at approximately the same time of day, all rats were treated with their respective antibody or p75NTR-Fc fusion protein. Three hours later all animals were given an intra-articular injection of 0.3 mg MIA into one knee (ETF-PBS was injected into the contralateral knee).

### Randomisation of Treatment

Prior to the start of the study rats were weighed and each cage of two rats was randomly assigned to a treatment group so that the mean body weight of animals in each group were approximately equal. In addition to each rat being allocated to a particular treatment group further randomisation was also carried out so that either the left or right knee of each rat was injected with MIA (with the contralateral knee from each rat injected with ETFPBS). The allocation of treatment group and which knee received treatment for each rat was produced using a random number generator in Microsoft Excel for the Mac (Version 14.1.1). Personnel who had no contact with the animals carried out the randomisation procedure and allocation.

Two 7 ml polypropylene vials were labeled for each animal to denote the left or right knee (total of 88 vials). Two people (one scoring and checking to the master randomisation sheet and one aliquoting the solution for the intraarticular injection) prepared the 88 vials. The aliquoting was carried out in sequence so that the MIA vials were filled first followed with the remaining vials being filled with ETF-PBS (this was the contralateral knee vial for each animal). Throughout the study in vivo scientists were blind to the treatment status of all animals.

### Animal Procedures

### Intra-Articular Injection of the Knee

All rats were anaesthetised by inhalation of Isoflurane using a Boyles Apparatus. The hairs on both knees of each animal were clipped and the knees swabbed with ethanol. Each knee was injected through the infra-patellar ligament with 50 μl of either 0.3 mg MIA in ETF-PBS or ETF-PBS alone using a 0.5 ml sterile Becton Dickinson Micro-Fine insulin syringe with an attached 27 G needle.

### Assessment of Spontaneous Pain

Spontaneous pain was determined for each animal by measuring the weight bearing of the left and right hind limbs using an incapacitance tester (Linton Instruments, U.K.). Rats were placed in an appropriately sized perspex animal box on the incapacitance tester so that their hind feet sat on separate sensors. The size of the box allowed the rat to sit comfortably without squashing but similarly did not permit it sufficient space to turn around. Once the rat was steady and calm, the weight bearing of each limb was recorded over 5 seconds and the average force in grams exerted by both hind limbs was recorded. The weight distribution of the hind paws was determined five times (the validity for which we have demonstrated previously) for each rat at each time point, and the mean of the five readings calculated. The individual weight bearing data was converted into a weight distribution by dividing the weight of the right limb by the total weight for both hind limbs.

### Spontaneous Pain Measurements Following MIA-Induced OA

Spontaneous pain was assessed using an incapacitance tester to measure the distribution of weight through the rear limbs. Assessments were carried out at baseline and at 3 weeks post-treatment with MIA.

It is evident from these studies that p75NTR-Fc is analgesic in the MIA rat model of OA. The analgesic effects p75NTR-Fc were greater that observed for anti-NGF antibodies (PG-007: biosimilar Pfizer anti-NGF antibody Tanezumab) at similar doses: 3 mg/kg subcutaneous.

1) a p75NTR neurotrophin binding protein (NBP)-Fc fusion protein, comprising:
(a) a p75NTR(NBP) portion selected from one of Seq ID no 1-19; and
(b) an immunoglobulin Fc portion selected from one of Seq ID no 20-24;
wherein, the p75NTR(NBP) and Fc portions are connected via a linker, the linker comprises a peptide of formula Gₓ, where x is 1, 2, 3, 4, 5 or 6.

2) The p75NTR(NBP)-Fc fusion protein according to claim 1 wherein the linker is GGG.

3) The p75NTR(NBP)-Fc fusion protein according to claim 1 or 2 wherein the Fc region is an Fc suitable for human use.

4) Use of the p75NTR(NBP)-Fc fusion protein as described in claims 1-3 for the treatment of pain.

5) A nucleic acid molecule encoding the p75NTR(NBP)-Fc fusion protein as described in claims 1-3.

6) A replicable expression vector for transfecting a cell, the vector comprising the nucleic acid molecule described in claim 5.

7) Use of the nucleic acid molecule as described in claim 5 or the vector as described in claim 6 for the treatment of pain.

8) Use of the p75NTR(NBP)-Fc fusion protein as described in claims 1-3 the nucleic acid molecule as described in claim 5, or the vector as described in claim 6 for use in the treatment of osteoarthritis.

9) A pharmaceutical composition, comprising the p75NTR(NBP)-Fc fusion protein as described in claims 1-3 the nucleic acid molecule as described in claim 5, or the vector as described in claim 6, and a pharmaceutically acceptable carrier and/or an excipient.


SEQUENCE LISTING (24 sequences)

SEQ ID NO: 1 - protein, 209 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDTSVPTSNDPDVPPDNEIIASTVAGVVTTVMG

SEQ ID NO: 2 - protein, 209 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDTSLPTSNDPDVPPDNEIIASTVAGVVTTVMG

SEQ ID NO: 3 - protein, 209 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDTSLPTSNDPDLPPDNEVIASTVAGVVTTVMG

SEQ ID NO: 4 - protein, 209 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDTSLPTSNDPDVPPDNEVIASTVAGVVTTVMG

SEQ ID NO: 5 - protein, 209 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDTSLPTSNDPDLPPDNEIIASTVAGVVTTVMG

SEQ ID NO: 6 - protein, 209 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDTSLPTSNDPDIPPDNEVIASTVAGVVTTVMG

SEQ ID NO: 7 - protein, 209 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDTSVPTSNDPDLPPDNEIIASTVAGVVTTVMG

SEQ ID NO: 8 - protein, 209 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDTSVPTSNDPDVPPDNEVIASTVAGVVTTVMG

SEQ ID NO: 9 - protein, 209 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDTSVPTSNDPDLPPDNEVIASTVAGVVTTVMG

SEQ ID NO: 10 - protein, 209 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDTSVPTSNDPDIPPDNEIIASTVAGVVTTVMG

SEQ ID NO: 11 - protein, 209 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDTSVPTSNDPDIPPDNEVIASTVAGVVTTVMG

SEQ ID NO: 12 - protein, 209 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDTSIPTSNDPDVPPDNEIIASTVAGVVTTVMG

SEQ ID NO: 13 - protein, 209 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDTSIPTSNDPDLPPDNEIIASTVAGVVTTVMG

SEQ ID NO: 14 - protein, 209 aa, synthetic construct; identical to SEQ ID NO: 12

SEQ ID NO: 15 - protein, 209 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDTSIPTSNDPDIPPDNEIIASTVAGVVTTVMG

SEQ ID NO: 16 - protein, 209 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDTSIPTSNDPDIPPDNEVIASTVAGVVTTVMG

SEQ ID NO: 17 - protein, 209 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDTSIPTSNDPDLPPDNEVIASTVAGVVTTVMG

SEQ ID NO: 18 - protein, 209 aa, synthetic construct; identical to SEQ ID NO: 16

SEQ ID NO: 19 - protein, 209 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDTSIPTSNDPDVPPDNEVIASTVAGVVTTVMG

SEQ ID NO: 20 - protein, 27 aa, synthetic construct
PKSCDKTHTCPPCPAPELLGGPSVFLF

SEQ ID NO: 21 - protein, 324 aa, synthetic construct
APSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 22 - protein, 390 aa, synthetic construct
SLEDTAVIPLFSECKAPKEDDVVSLACLVKGYFPEPVQVTWEPEMQNQKPWTFPAMKKGQEYIHVFSLTTWWKPGSHSCTVHHKASSFRKKMTFQEPASWAPQRTSALPVTSKEPTPAPTTLRKSEPSTRHTQPETQKPRIPVDTPLKECQSHTHPPSIYLLHPPLQGLWLKGEATFTCLVVGDDLKDAHLSWELSERSNGMFVESGPLEKHTNGSQSRSSRLALPRSSWAMGTSVTCKLSYPNLLSSMEVVGLKEHAASAPRSLTVHALTTPGLNASPGATSWLQCKVSGFSPPEIVLTWLEGQREVDPSWFATARPTAQPGNTTFQTWSILLVPTIPGPPTATYTCVVGHEASRQLLNTSWSLDTGGPSHGSSSGSRAGQPQETSSHA

SEQ ID NO: 23 - protein, 455 aa, synthetic construct
ESSSSPTLFPLVSCESSDESQVALGCLAHGFLPDSIKFSWDYKNNSAIDIGKYKTFPSVLQEGKYLASSQVLLPSVDVLQDTEDYLMCKVQHPKENKNLKVPFPGPIDPISPNVTVYIPPRDSFSGQPRTSKLSCWATGFSPKQISLVWLRDGKPVLSGFTTGEAEPEPKPSEPRSQTFQINSMLTISESDWLNQIVFTCVARHQGEEVMKNVSSVCGPSQSPSINIFTTPPSFAGIFLTKSAKLSCLVTGLATYESLNISWTRENGEALKTDLLYSESFPNATFSVTGKATVCLEDWESGEKFMCIVTHTDLPSPLKQTMSKPKVVKQQPAVYLMPPTREQLSLRESASITCLVKAFSPPDVFVQWLQKGQPVASDSYVTSNPMPEPQTKDLYFAYSILNVSEEEWSAGDTFTCVVGHEALPHLVTERTVDKSTGKPSLYNVSLVMSDTANTCF

SEQ ID NO: 24 - protein, 474 aa, synthetic construct
MESVLYWVFLVAILKGVQGDVQLVESGGDLVKPGGSLRLSCVASGFTFSSCAMSWVRQSPGKGPQWVATIRYDGSDIYYADAVKGRFSISRDNAKNTVYLQMNSLRAEDTAVYYCAKAPPYDSYHYGMDYWGPGTSLFVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK